(19) United States
(12) Patent Application Publication
(10) Pub. No.: US 2006/0014275 A1
(43) Pub. Date: Jan. 19, 2006

(54) Cell culture carrier and jig for cell culture

(75) Inventors: Takashi Yanagisawa, Tokyo (JP); Izumi Matsumoto, Tokyo (JP)

Correspondence Address: BIRCH STEWART KOLASCH & BIRCH, PO BOX 747, FALLS CHURCH, VA, 22040-0747, US

(73) Assignee: GSI Creos Corporation, Tokyo (JP)

(21) Appl. No.: 11/127,270

(22) Filed: May 12, 2005

(30) Foreign Application Priority Data
Feb. 21, 2005 (JP) 2005-44814
Sep. 26, 2003 (JP) 2003-336089

(51) Int. Cl. C12M 1/22

(52) U.S. Cl. 435/305.2

(57) ABSTRACT

The present invention provides a cell culture carrier and a method for cell culture, which are capable of growing multi-layered cells or stably growing cells with a high density. To solve the above described disadvantage, the cell culture carrier of the present invention comprises cup-stacked type carbon nano tubes produced by a catalytic chemical vapor deposition method, a plurality of bottomless cup-shaped carbon layers (graphene sheet) are stacked in each of the cup-stacked type carbon nano tubes, and edges of the stacked carbon layers are exposed.

### BACKGROUND OF THE INVENTION

1. Field of the Invention

The present invention relates to a cell culture carrier and a jig for cell culture.

2. Related Art

Antibodies and proteins are purified to study and develop new medicines, but it is difficult to purify them in animal bodies, thus cultured cells, which are cultured and grown outside of living organisms, have been studied these days. In western countries, using experimental animals is significantly restricted for animal protection, so that cultured cells are now in strong demand.

Antibodies and proteins are produced by using the cultured cells so as to examine bioactivity and toxicity of medicines.

Some cells, especially cells derived from human bodies, have adherering dependency or adhere to living organisms to grow, so they cannot survive outside of living organisms for a long time; it is difficult to mass-culture the cells.

To solve the problem, several cell culture carriers have been developed.

For example, in Japanese Patent Gazette No. 8-9960, patterns are formed on a surface of a substrate so as to vary ease of bonding cells, and the cells are cultured thereon so that patterning of the cells can be performed.

However, the substrate (the cell culture carrier) has a following disadvantage.

Namely, the cells grow and increase along the surface of the substrate, but they grow thereon as a mere single layer.

Cells existing in a living organism constitute a complex multi-layered structure, so a bioactive test, etc., cannot be performed under conditions similar to the living organism with the single-layered cells.

### SUMMARY OF THE INVENTION

An object of the present invention is to provide a cell culture carrier and a method for cell culture, which are capable of growing multi-layered cells or stably growing cells with a high density.

To solve the above described disadvantage, the cell culture carrier of the present invention comprises cup-stacked type carbon nano tubes produced by a catalytic chemical vapor deposition method, a plurality of bottomless cup-shaped carbon layers (graphene sheet) are stacked in each of the cup-stacked type carbon nano tubes, and edges of the stacked carbon layers are exposed.

In the cell culture carrier, the cup-stacked type carbon nano tubes may be entangled in a three dimensional space.

In the cell culture carrier, each of the cup-stacked type carbon nano tubes may have a hollow shape having no bridge.

In the cell culture carrier, the edges of the carbon layers, which constitute an inner face and an outer face of the hollow shape of the cup-stacked type carbon nano tube, may be exposed.

In the cell culture carrier, area of exposing the edges of the carbon layers may be 2% or more of area of an outer face of the cup-stacked type carbon nano tube.

In the cell culture carrier, the exposed edges of the carbon layers may be irregularly formed, and a part of a surface of the cup-stacked type carbon nano tube, in which the edges of the carbon layers are exposed, may include asperities at the atomic level.

In the cell culture carrier, the cup-stacked type carbon nano tubes are not graphitized even if they are heat-treated at temperature of 2,500° C. or more.

In the cell culture carrier, a D-peak (1360 cm⁻¹) of a raman spectrum does not disappear even if the cup-stacked type carbon nano tubes are heat-treated at temperature of 2,500° C. or more.

Another cell culture carrier is made of a carbon composite body including cup-stacked type carbon nano tubes, the cup-stacked type carbon nano tubes are produced by a catalytic chemical vapor deposition method, the carbon composite body is made by baking a mixed material including the cup-stacked type carbon nano tubes, in each of which a plurality of bottomless cup-shaped carbon layers are stacked and edges of the stacked carbon layers are exposed in a surface, and resin so as to carbonize, and a surface of the carbon composite body is treated so that parts of the cup-stacked type carbon nano tubes are exposed in the surface.

In the cell culture carrier, the surface of the carbon composite body may be heated and oxidized in an oxidizing atmosphere so as to expose the cup-stacked type carbon nano tubes in the surface.

In the cell culture carrier, content of the cup-stacked type carbon nano tubes with respect to the mixed material may be 30-90 wt %.

Further, another cell culture carrier is made of a carbon composite body, wherein the carbon composite body is made by baking a mixed material including fine activated carbon powders and resin so as to carbonize the resin, and a surface of the carbon composite body is treated so that a part of the activated carbon is exposed in a surface.

In the cell culture carrier, the surface of the carbon composite body may be heated and oxidized in an oxidizing atmosphere so as to expose the activated carbon in the surface.

In the cell culture carrier, content of the activated carbon with respect to the mixed material may be 30-90 wt %.

A cell culture jig of the present invention comprises a cell culture carrier, the cell culture carrier is made of a carbon composite body including cup-stacked type carbon nano tubes, the cup-stacked type carbon nano tubes are produced by a catalytic chemical vapor deposition method, the carbon composite body is made by baking a mixed material including the cup-stacked type carbon nano tubes, in each of which a plurality of bottomless cup-shaped carbon layers are stacked and edges of the stacked carbon layers are exposed in a surface, and resin so as to carbonize, a surface of the carbon composite body is treated so that parts of the cup-stacked type carbon nano tubes are exposed in the surface, and the cell culture carrier is fixed to a part, which contacts a culture solution and cells.

Another jig comprises a cell culture carrier, the cell culture carrier is made of a carbon composite body, wherein the carbon composite body is made by baking a mixed material including fine activated carbon powders and resin so as to carbonize the resin, a surface of the carbon composite body is treated so that a part of the activated carbon is exposed in a surface, and the cell culture carrier is fixed to a part, which contacts a culture solution and cells.

### BRIEF DESCRIPTION OF THE DRAWINGS

FIG. 1 is a transmission electron microscope of a cup-stacked type carbon nano tube, which was produced by a catalytic chemical vapor deposition method and had a herringbone structure;

FIG. 2 is an enlarged view of FIG. 1;

FIG. 3 is a schematic view of FIG. 2;

FIG. 4 is a transmission electron microscope of a cup-stacked type carbon nano tube, which were heat-treated in the air for an hour at temperature around 530° C.;

FIG. 5 is an enlarged view of FIG. 4;

FIG. 6 is a further enlarged view of FIG. 5;

FIG. 7 is a schematic view of FIG. 6;

FIG. 8 shows raman spectra of a cup-stacked type carbon nano tube (sample number: 24PS), which were heat-treated in the air for an hour at temperature of 500° C., 520° C., 530° C. and 540° C.;

FIG. 9 shows raman spectra of cup-stacked type carbon nano tubes of sample number 19PS and 24PS, in which edges of carbon layers were exposed by the heat treatment;

FIG. 10 shows raman spectra of the cup-stacked type carbon nano tubes of the sample number 19PS and 24PS, in which the edges of the carbon layers were exposed by the heat treatment and which were heated at temperature of 3000° C.;

FIG. 11 is graphs showing fiber lengths of cup-stacked type carbon nano tubes, which were ground by ball milling, with respect to elapsed time;

FIG. 12 is a transmission electron microscope of cup-stacked type carbon nano tubes before ball-milling;

FIG. 13 is a transmission electron microscope of the cup-stacked type carbon nano tubes after a lapse of two hours from the beginning of the ball milling;

FIG. 14 is a transmission electron microscope of the cup-stacked type carbon nano tubes after a lapse of five hours from the beginning of the ball milling;

FIG. 15 is a transmission electron microscope of the cup-stacked type carbon nano tubes after a lapse of 10 hours from the beginning of the ball milling;

FIG. 16 is a transmission electron microscope of the cup-stacked type carbon nano tubes after a lapse of 24 hours from the beginning of the ball milling;

FIG. 17 is a transmission electron microscope of the cup-stacked type carbon nano tube during the ball milling, wherein a cup-shaped carbon layer started to break off;

FIG. 18 is an enlarged view of FIG. 17;

FIG. 19 is a further enlarged view of FIG. 18;

FIG. 20 is a transmission electron microscope of a cup-stacked type carbon nano tube constituted by stacking several dozen of bottomless cup-shaped carbon layers, wherein one of the carbon layers left;

FIG. 21 is a schematic view of the carbon layer, in which calboxyl groups are modified to the exposed end;

FIG. 22 is a sectional view of a jig for cell culture (a well plate);

FIG. 23 is an explanation view of a jig for cell culture (a well plate) having slits;

FIG. 24 is an explanation view of particle-shaped cell culture carriers;

FIG. 25 is an explanation view of a cell culture carrier fixed by an O-ring;

FIG. 26 is an explanation view of a cell culture carrier fixed by a net-shaped member;

FIG. 27 is a scanning electron microscope of a surface of the cell culture carrier;

FIG. 28 is an explanation view of cell culture carriers fixed on a resin plate or film;

FIG. 29 is a graph of number of cells, which were cultured in EXPERIMENT 2, after a lapse of five days;

FIG. 30 is a micrograph of cells, which were cultured with a sample 24-OXSL, after a lapse of five days;

FIG. 31 is a micrograph of cells, which were cultured in a culture solution including no cup-stacked type carbon nano tubes, after a lapse of five days;

FIG. 32 is a graph of number of cells, which were cultured in EXPERIMENT 2, after a lapse of 12 days; and

FIG. 33 is a graph of number of cultured cells with respect to culture-days.

### DESCRIPTION OF THE PREFERRED EMBODIMENTS

Preferred embodiments of the present invention will now be described in detail with reference to the accompanying drawings.

Firstly, a cup-stacked type carbon nano tube (CNT) of the present invention, which is used for a cell culture carrier, will be explained.

The cup-stacked type CNT is produced by a catalytic chemical vapor deposition method and has a herringbone structure in which bottomless cup-shaped carbon layers are stacked, and edges of the stacked carbon layers of the cup-stacked type CNT are exposed.

Many of the cup-stacked type CNTs are entangled and formed like a net so as to used as a cell culture carrier. Preferably, the cup-stacked type CNTs are entangled in a three dimensional space. With this structure, cells can be three-dimensionally cultured and grown and can form a multilayered structure.

Natures of the cup-stacked type CNT will be explained.

An example of methods of producing the cup-stacked type CNT will be explained.

A known vertical reactor was used. Note that, a horizontal reactor may be used.

Benzene, which was a basic material, was introduced into a chamber of the reactor by a hydrogen flow whose flow rate is 0.3 l/h with partial pressure corresponding to steam pressure of 20° C. Ferrocene, which was used as a catalytic agent, was vaporized at temperature of about 185° C. and introduced into the chamber with concentration of about 3×10⁻⁷ mol/s. Reaction temperature was about 1100° C. and reaction time was about 20 minutes, so that cup-stacked type CNTs, which had herringbone structures and whose average diameter was about 100 nm, were produced. Many bottomless cup-shaped carbon layers could be stacked by adjusting the flow rate of the basic material and the reaction temperature (they depend on a size of the reactor), so that hollow cup-stacked type CNTs, which had no bridges and whose sizes were several dozen nm to several dozen μm, were produced.

Next, the cup-stacked type CNT will be explained.

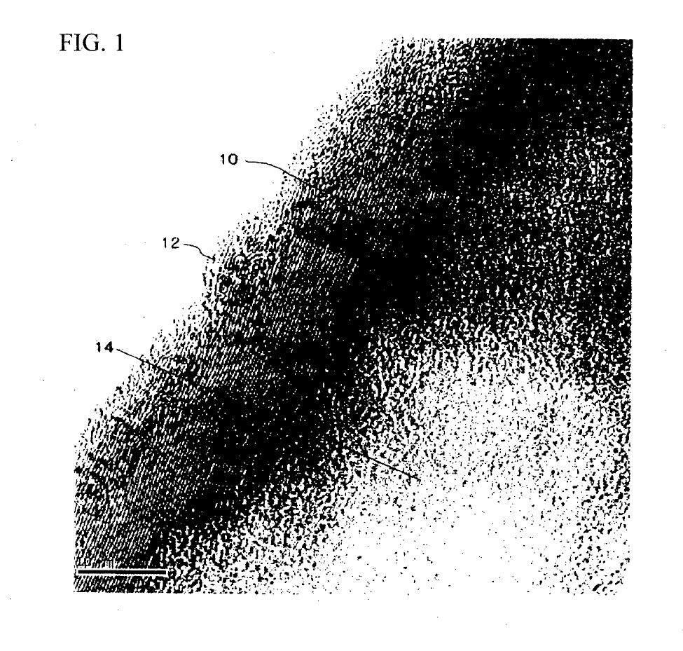

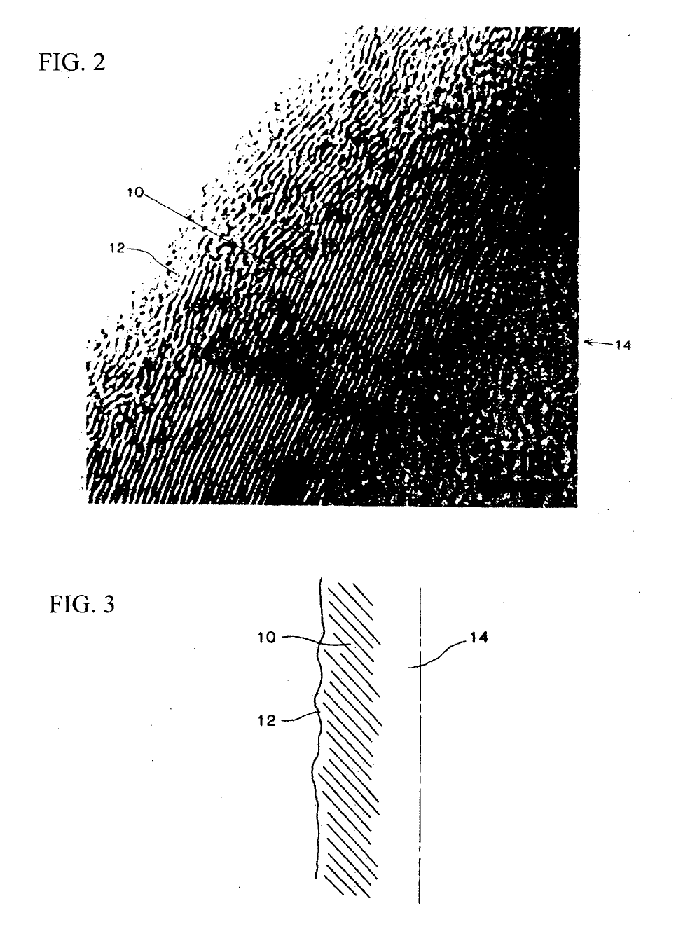

FIG. 1 is a transmission electron microscope of the cup-stacked type CNT, which was produced by a catalytic chemical vapor deposition method; FIG. 2 is an enlarged view thereof; and FIG. 3 is a schematic view thereof.

According to the drawings, a deposit layer 12, which was formed by depositing surplus amorphous carbon, covers an inclined carbon layer 10. A symbol 14 stands for a center hole.

The cup-stacked type CNT, which had the deposit layer 12, was heat-treated in the air at temperature of 400° C. or more, preferably 500° C. or more, more preferably 520° C.-530° C., for one to several hours, so that the deposit layer 12 was oxidized, and removed, and the edge of the carbon layer (the edge of hexagonal carbon layer) was partially exposed.

In another case, the deposit layer 12 can be removed and the end of the carbon layer can be exposed by cleaning the cup-stacked type CNT with supercritical water.

Further, the deposit layer 12 can be removed by soaking the cup-stacked type CNT in hydrochloric acid or sulfuric acid, agitating with a stirrer and heating until reaching temperature of about 80° C.

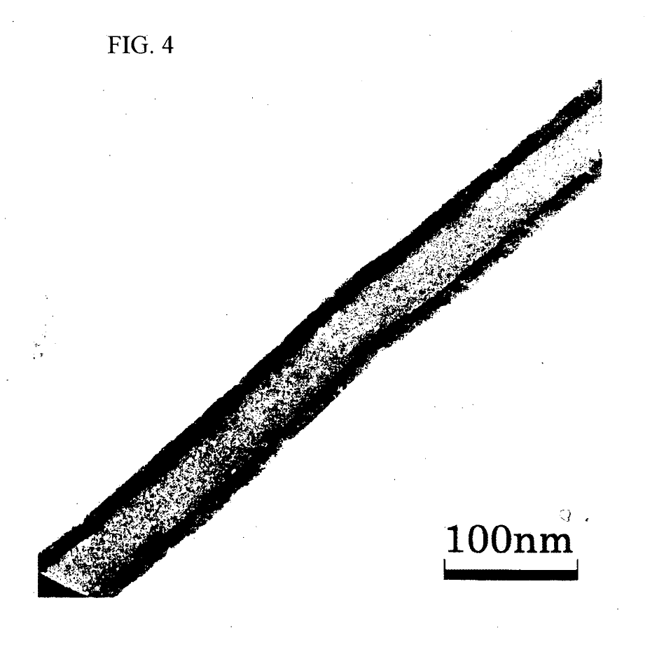

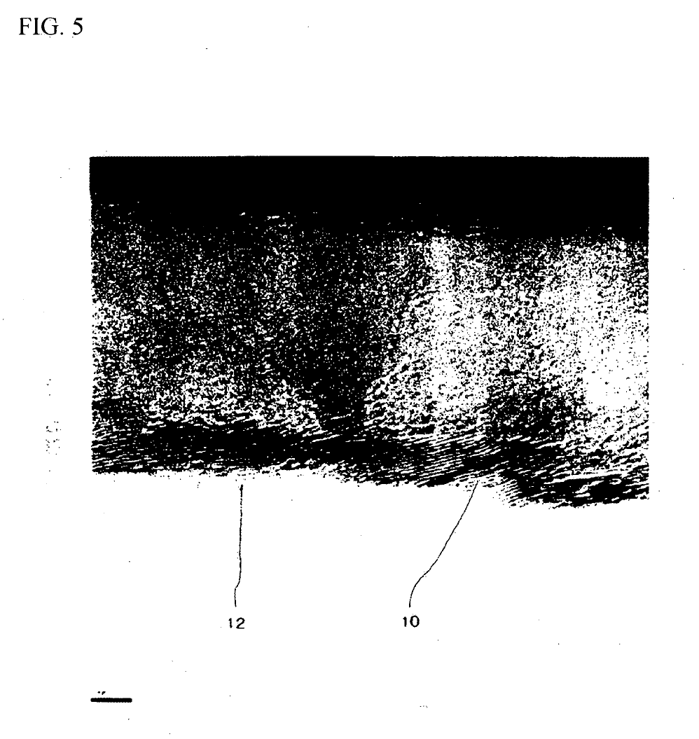

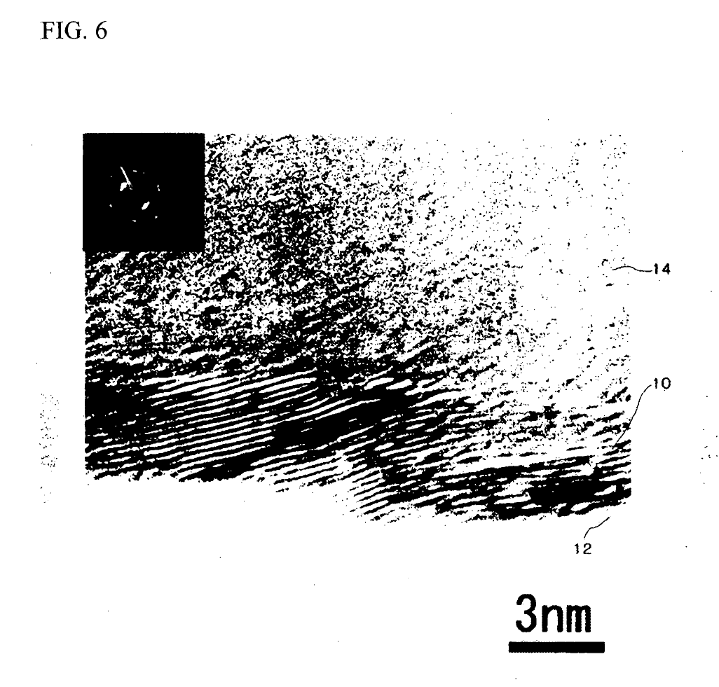

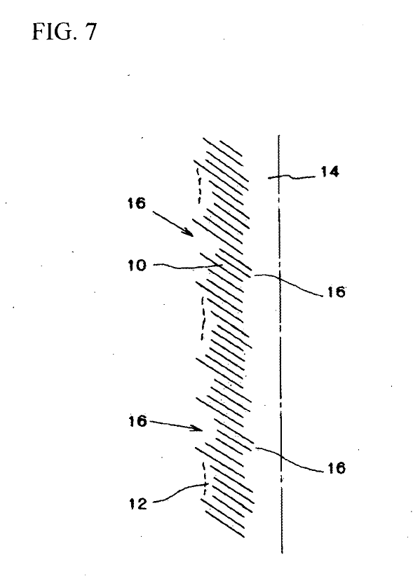

FIG. 4 is a transmission electron microscope of a cup-stacked type carbon nano tube, which were heat-treated in the air for an hour at temperature around 530° C.; FIG. 5 is an enlarged view thereof; FIG. 6 is a further enlarged view thereof; and FIG. 7 is a schematic view thereof.

According to FIGS. 5-7, the deposit layer 12 was partially removed by the above described heat treatment, so that an edge of the carbon layer 10 (an end of a carbon six-member ring) was exposed. The rest deposit layer 12 was almost decomposed and merely stuck. Note that, 100% of the deposit layer 12 can be removed by further cleaning with supercritical water.

According to FIG. 4, many bottomless cup-shaped carbon layers were stacked, and the cup-stacked type CNT 10 was hollow within a range of at least several dozen nm to several hundred nm.

Inclination angles of the carbon layers were about 15-25° According to FIGS. 6 and 7, exposed parts of an outer face and an inner face, in which the edge of the carbon layer 10 was exposed, included fine asperities 16 at the atomic level. As shown in FIG. 2, the asperities 16 were not seen before removing the deposit layer 12, but they appeared by removing the deposit layer 12.

The edges of the carbon layers 10 easily combined with other atoms and had high degree of activity. The reason is that an oxygen-containing functional group, e.g., phenoric hydroxyl group, calboxyl group, quinone carbonyl group, increases at the exposed edge of the carbon layer when the deposit layer 12 is removed by the heat treatment in the air, and the oxygen-containing functional group has high hydrophilic property and high affinity to various substances.

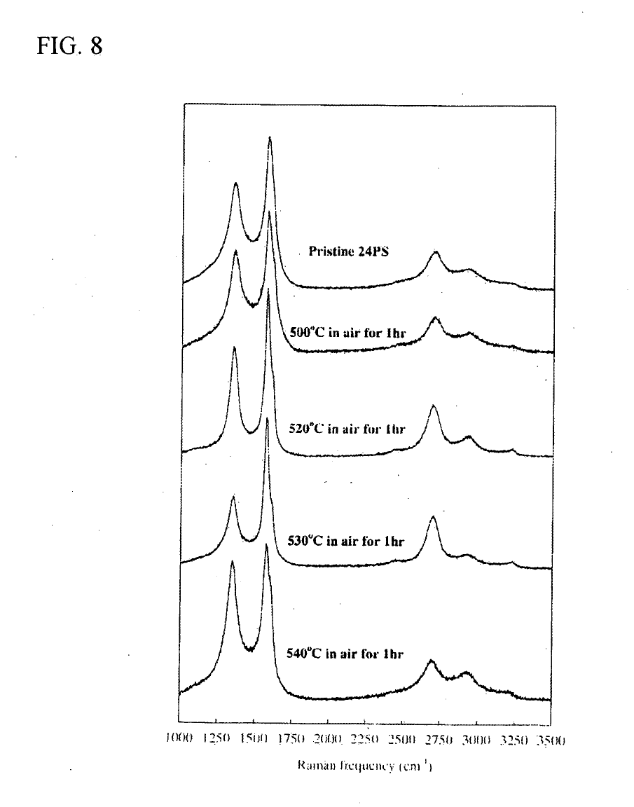

FIG. 8 shows raman spectra of a cup-stacked type CNT (sample number: 24PS), which were heat-treated in the air for an hour at temperature of 500° C., 520° C., 530° C. and 540° C.

As shown in FIGS. 5-7, the deposit layers 12 were removed the heat treatment; according to the raman spectra shown in FIG. 8, D-peaks (1360 cm⁻¹) and G-peaks (1580 cm⁻¹) were observed, so the results indicated that the sample was the cup-stacked type CNT but was not graphitized.

Namely, we think that the cup-stacked type CNT had a grind turbostratic structure, in which surfaces of carbon layers were misaligned.

In the cup-stacked type CNT having the grind turbostratic structure, surfaces of hexagonal carbon layer were stacked in parallel but misaligned in the horizontal direction or turned, so it has no crystallographic regularity.

By the turbostratic structure, intercalation to other atoms, etc. between layers is restricted. This is an advantageous point. Since substances are restricted to enter another layer, atoms, etc. are easily supported at exposed edges of the carbon layers, which has high degree of activity, so that it will be used as an efficient carrier.

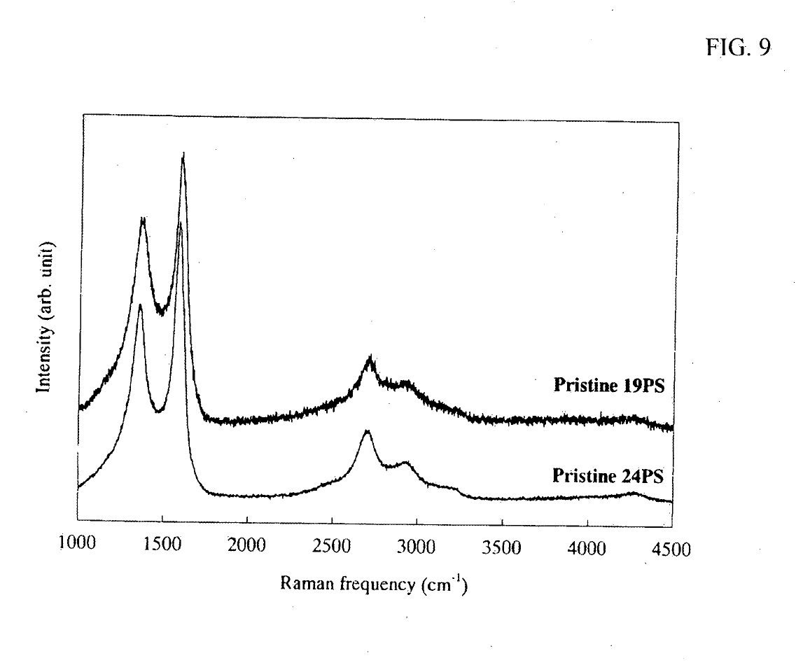

FIG. 9 shows raman spectra of cup-stacked type CNTs of sample number 19PS and 24PS, in which edges of carbon layers were exposed by the heat treatment.

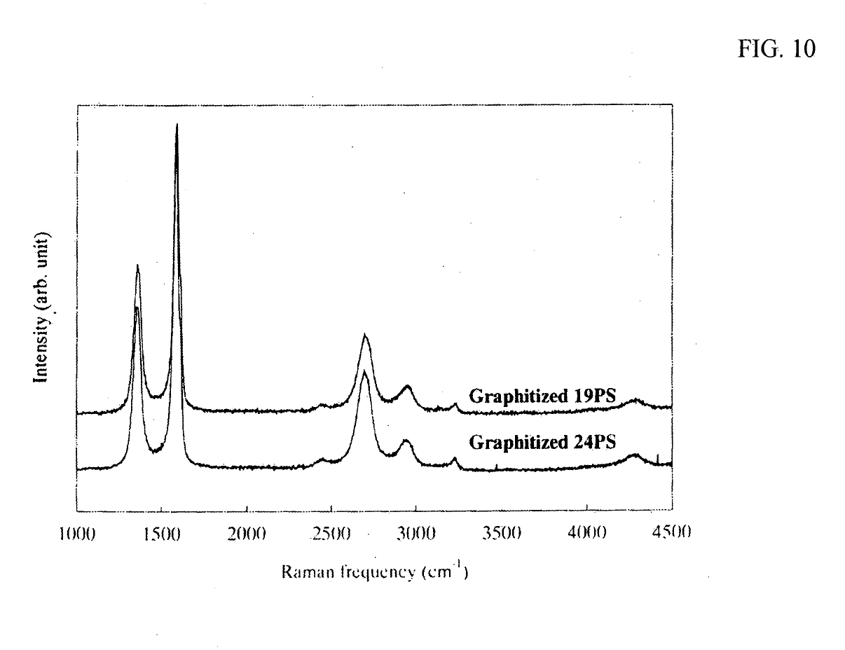

FIG. 10 shows raman spectra of the cup-stacked type CNTs of the sample number 19PS and 24PS, in which the edges of the carbon layers were exposed by the heat treatment (ordinary graphitizing treatment) and which were heated at temperature of 3000° C.

According to FIG. 10, D-peaks were not disappeared by heating the cup-stacked type CNTs, in which the edges of the carbon layers were exposed. This results indicates that they are not graphitized by the ordinary graphitizing treatment.

By X-ray diffraction not shown, diffraction lines of face (112) were not observed, so the cup-stacked type CNTs were not graphitized.

We think that the deposit layers 12, which are easily graphitized, were removed, so that the samples were not graphitized by the ordinary graphitizing treatment. Further, the left parts of the herringbone structures were not graphitized.

Since the cup-stacked type carbon nano tubes are not graphitized in a high temperature atmosphere, they are thermally stable.

The above described cup-stacked type CNT having the herringbone structure is a short fiber (length: several dozen μm), which is formed by stacking tens to hundreds of thousands of unit carbon layers each of which has the bottomless cup shape. The short fiber has a large molecular weight (length) and is insoluble.

The cup-stacked type CNT may be divided into short fibers, each of which is formed by stacking several to several hundred of the unit carbon layers.

For example, the cup-stacked type CNT may be divided into the short fibers by adding water or a solvent in a mortar and slowly grinding with a pestle.

Namely, the cup-stacked type CNTs, which have the deposit layers 12 or from which the deposit layers 12 have been partially or completely removed, are put in the mortar and slowly mechanically ground with the pestle, so that the carbon nano tubes are ground and formed into short fibers.

By controlling the grind treatment in the mortar on the basis of experiences, the cup-stacked type CNTs, each of which is formed by stacking several to several hundred of the unit carbon layers, can be produced.

Strength of the ring-shaped carbon layers are relatively high, and the carbon layers are mutually combined by small van der waals force; the ring-shaped carbon layers are not broken, but they are separated at weakly combined sections.

Preferably, the cup-stacked type CNTs in the mortar are ground in liquid nitrogen. When the liquid nitrogen evaporates, moisture in the air is absorbed and formed into ice and the cup-stacked type CNTs are ground together with the ice, so that mechanical stress can be reduced and the fibers can be separated at positions between the unit layers.

In industrial fields, the cup-stacked type CNTs may be ground by ball-milling.

An example of adjusting lengths of the cup-stacked type CNTs by ball-milling will be explained.

A ball mill manufactured by Asahi Rika Seisakujo was used.

Used balls were made of alumina and had diameters of 5 mm. 1 g of the cup-stacked type CNTs, 200 g of the alumina balls and 500 cc of distilled water were put in a cell and rotated at a rotational speed of 350 rpm, and samples were measured after lapse of 1, 3, 5, 10 and 24 hours.

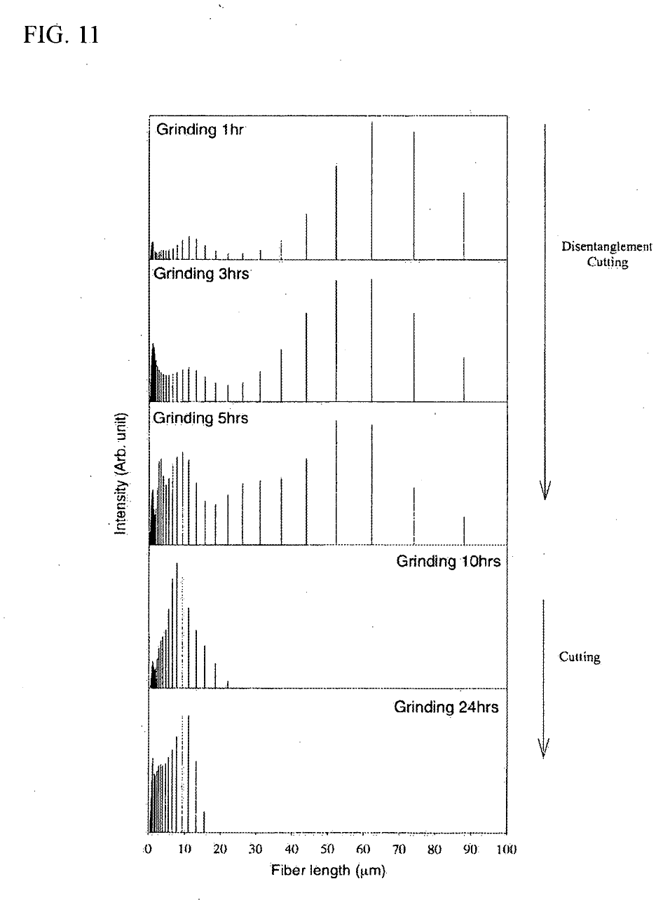

FIG. 11 is graphs showing fiber lengths of cup-stacked type CNTs, which were measured by a laser granulometry meter, with respect to elapsed time.

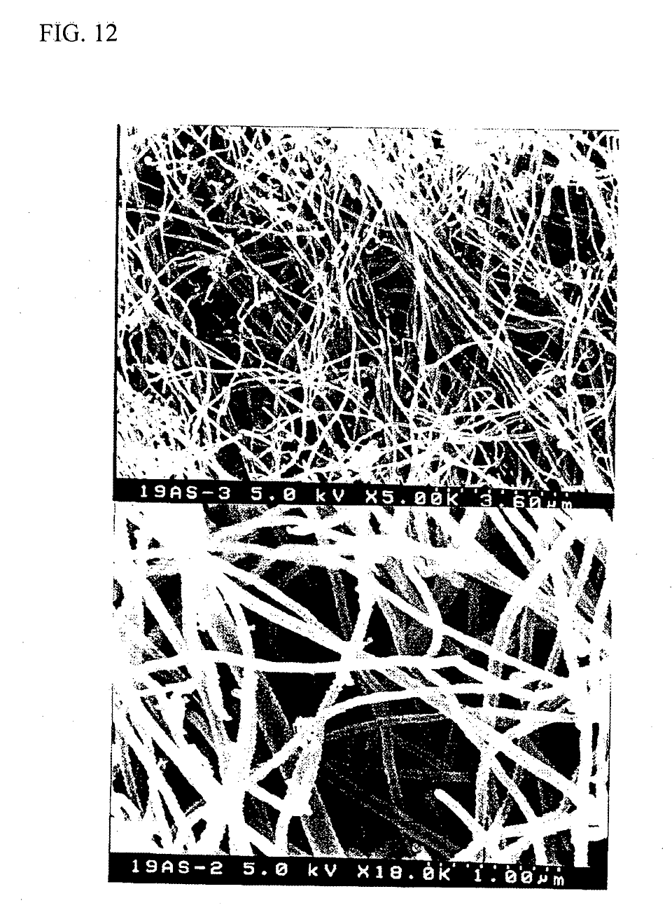

According to FIG. 11, the fiber lengths were shortened with the lapse of milling time. Especially, after the lapse of 10 hours, the fiber lengths were suddenly shortened to 10 μm or less. After the lapse of 24 hours, other peaks appeared around 1 μm, so the fibers were made thinner. The reason of appearing the peaks around 1 μm is that lengths and diameters of the fibers were equal, so the diameters of the fibers were double-counted. This is clearly shown in the micrographs of FIGS. 12-16.

FIG. 12 shows cup-stacked type CNTs before milling, the cup-stacked type CNTs, whose lengths were several dozen μm, were entangled, and apparent density was very low.

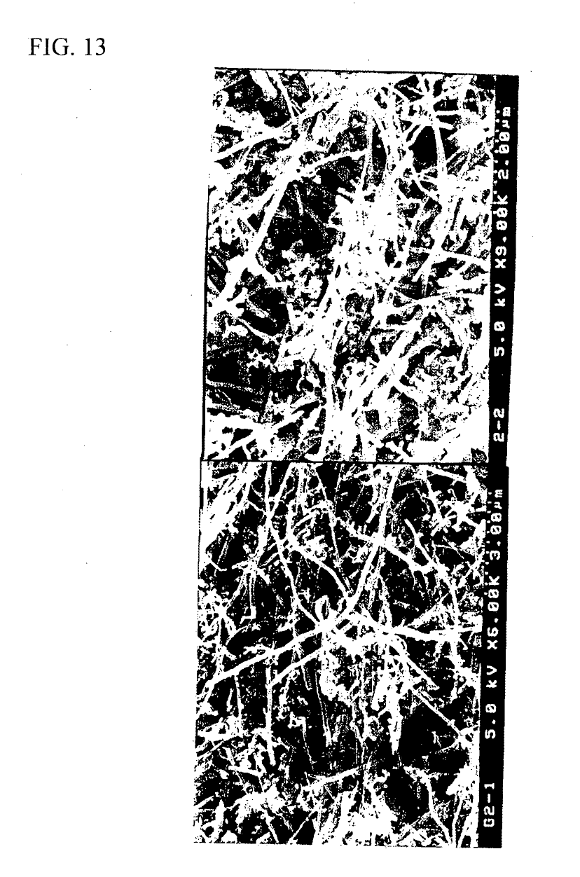

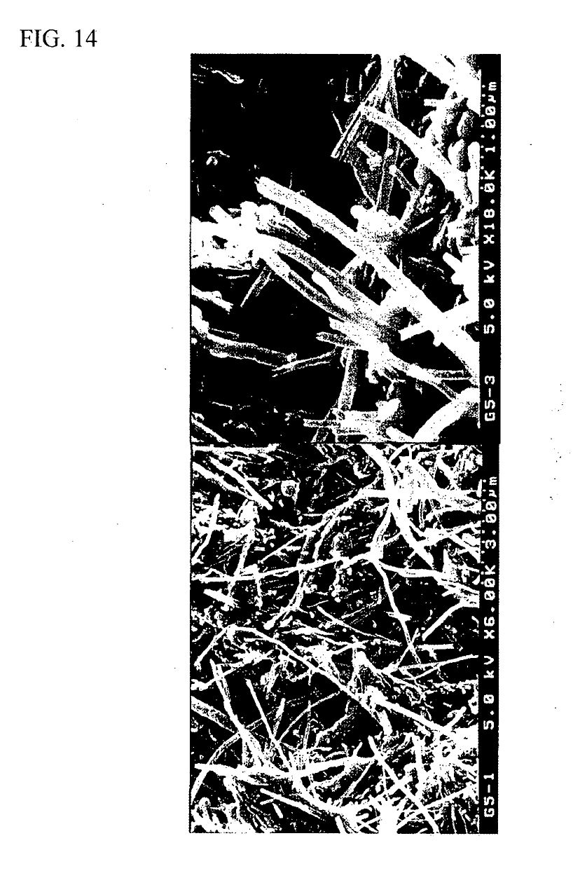

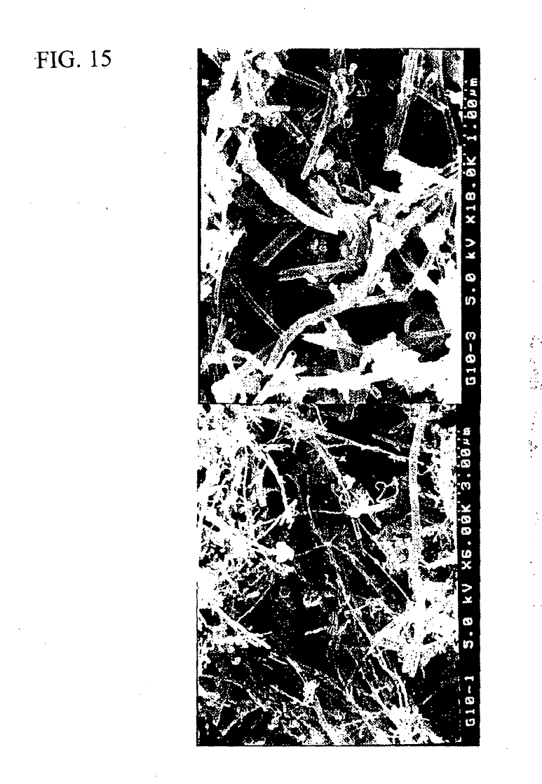

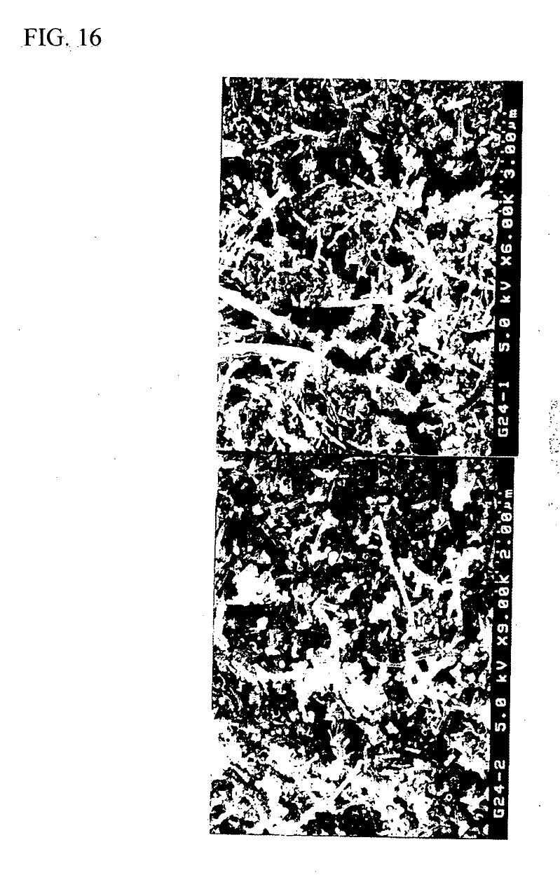

The lengths were shortened with the lapse of two hours (FIG. 13), five hours (FIG. 14), 10 hours (FIG. 15) and 24 hours (FIG. 16), the fibers were formed into particles after the lapse of 24 hours, the entanglement of the fibers were not observed, and the apparent density was high.

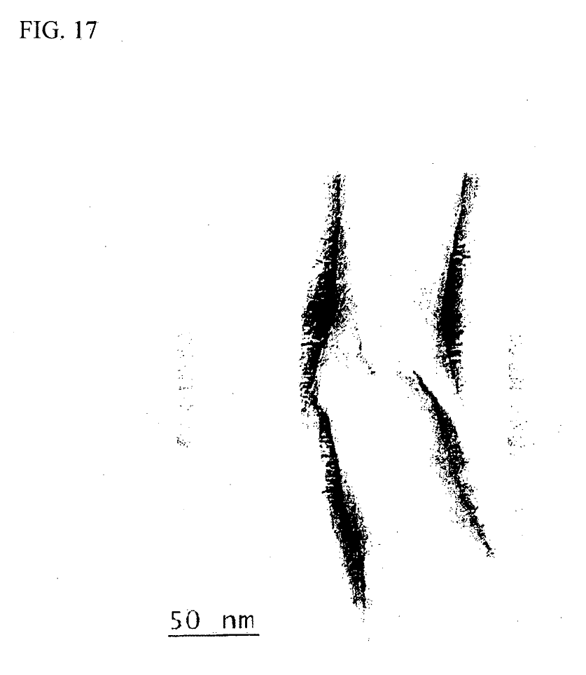

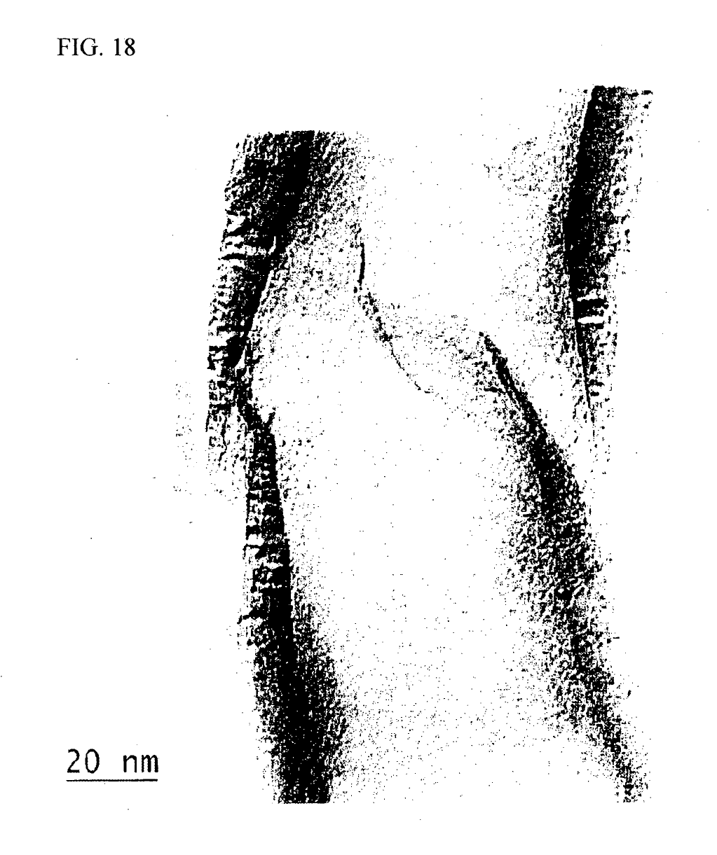

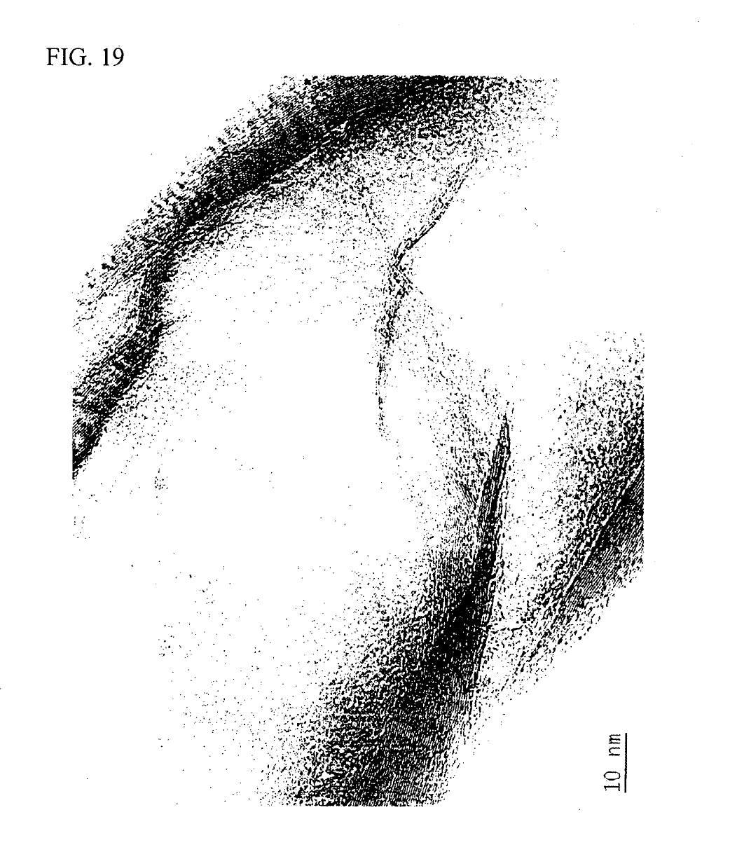

In transmission electron microscopes of FIGS. 17-19, the cup-stacked type CNT was just separated while the milling process. FIGS. 18 and 19 are enlarged views of FIG. 17.

As clearly shown in the drawings, the separation of division of the cup-stacked type CNT was performed by pulling out the bottomless cup-shaped carbon layers.

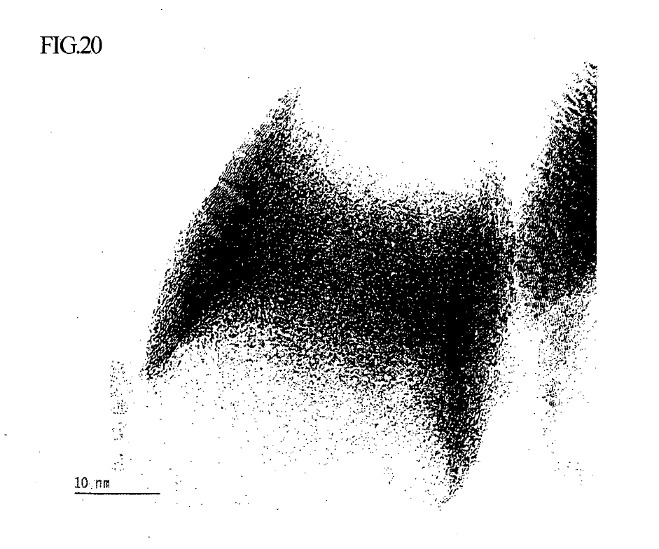

FIG. 20 is a transmission electron microscope of the interesting cup-stacked type CNT, which were constituted by stacking several dozen of bottomless cup-shaped carbon layers to adjust its length. It is formed into a hollow shape having no bridges. Edges of the carbon layers, which constitute an inner face and an outer face of the hollow shape, were exposed. The length of the cup-stacked type CNT may be optionally adjusted by changing milling conditions.

The cup-stacked type CNT shown in FIG. 20 had a thin tube-shape having a large hollow space, and its length and diameter were about 60 nm.

The bottomless cup-shaped carbon layers were pulled out and separated each other, and their shapes were not broken.

On the other hand, if ordinary carbon nano tubes, which are concentrically formed, are ground, the tubes are broken, cracks are formed in their outer faces in axial directions, agnails are formed and coreless states occur, so that it is difficult to adjust their lengths.

As described above, the exposed edges of the carbon layers 10 are easily combined with other atoms and have high degree of activity. The reason is that an oxygen-containing functional group, e.g., phenoric hydroxyl group, calboxyl group, quinone carbonyl group, increases at the exposed edge of the carbon layer when the deposit layer 12 is removed by the heat treatment in the air, and the oxygen-containing functional group has high hydrophilic property and high affinity to various substances.

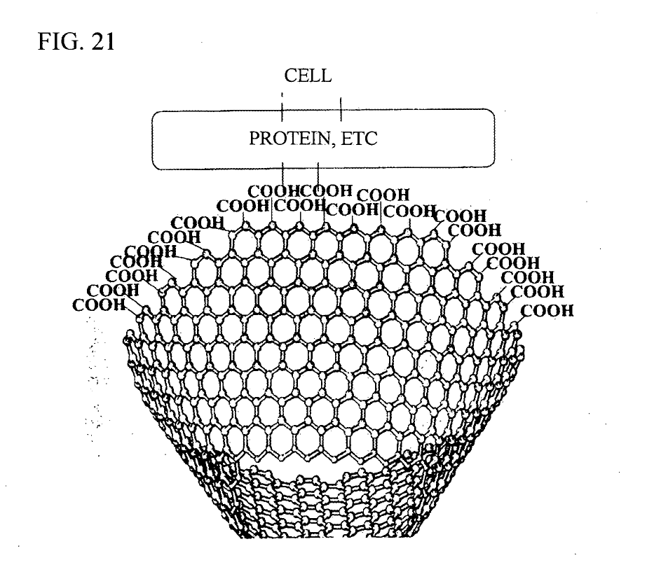

For example, if the exposed edge of the carbon layer 10 is modified with calboxyl groups, etc. as shown in FIG. 21, various proteins easily combine with the calboxyl groups.

Namely, various proteins, antibodies, vaccines, genes, etc. can be combined with the cup-stacked type CNTs, so the cup-stacked type CNTs can be used as carriers for the combined substances.

Conventionally, carriers, which are capable of carrying vaccines, antibodies, proteins and genes at the molecular level, are not realized, so the substances are locally administered with injection solutions or capsules.

On the other hand, in the cup-stacked type CNTs, the oxygen-containing functional groups, e.g., phenoric hydroxyl group, calboxyl group, quinone carbonyl group, increase at the exposed edges of the carbon layers, and the oxygen-containing functional groups have high hydrophilic property and high affinity to various substances; the exposed edges have high degree of activity and are capable of combining with the vaccines, antibodies, proteins, genes, etc. at the molecular level, and medicines can be administered and carried to very fine places, without surgical operations, by using metabolism of patients, further the cup-stacked type CNTs may be used as carriers for carrying object substances of gene remedy.

The cup-stacked type CNTs may be effectively used as carriers for cell culture.

Namely, the cell culture carrier may be produced by three-dimensionally entangling many of the cup-stacked type CNTs to form into a net (or a thin mat) or bonding many of the cup-stacked type CNTs on a surface of a micro bead (e.g., a plastic spherical body having diameter of 1-5 mm) to form into a spherical shape.

Cells are cultured by the steps of: accommodating the cell culture carrier in an incubator; supplying a culture medium solution in the incubator; disseminating cells on the carrier; and maintaining a prescribed temperature.

As shown in FIG. 12, the cup-stacked type CNTs 10 are three-dimensionally complexly entangled. As described above, the cup-stacked type CNTs have high degree of activity and are easily combined with other substances. The cup-stacked type CNTs, which have been previously combined with proteins, may be use for cell culture.

Molt-4 (lymphoblast) was cultured with the cell culture carriers, cell were well cultured and grown on the cup-stacked type CNTs, which acted as scaffolds, number of the cultured cells were much greater than that of cells cultured without adding the cup-stacked type CNTs, and the cells were efficiently cultured. By adding the cup-stacked type CNTs, the cells stably maintained spherical shapes, and colors of cellular cytoplasm were also stable.

On the other hand, some cells cultured without adding the cup-stacked type CNTs were broken, and cellular cytoplasm came out therefrom, so their cell activity were low.

Note that, other cells, e.g., liver cells, muscle cells, nerve cells, endocapillary cells, endocrine cells, skin/mucosal cells, other than Molt-4 (lymphoblast) may be cultured with the cell culture carrier.

While culturing cells, the cells produce infinitesimal metabolic products and metabolic wastes products, which have toxicities to themselves. In some cases, the metabolic products and metabolic wastes products must be removed so as to highly efficiently culture cells. Further, if nutrient contents are insufficient during culture, cells cannot be well cultured.

As described above, the cup-stacked type CNT of the present invention has high degree of activity and is formed into the hollow shape. The metabolic products and metabolic wastes products are adsorbed by the cup-stacked type CNT, and a circumstance of cell culture can be cleaned, so that cells can be cultured in the suitable circumstance. As described in EXPERIMENT 2, on the twelfth day or in the final stage of the culture, in which a culture solution must be supplemented or exchanged, reducing (or deadening) cells could be stopped, and numbers of living cells, which have been cultured with the cup-stacked type CNTs, were five to ten times greater than that of cells cultured without adding the cup-stacked type CNTs.

If nutrient contents, e.g., protein, are combined with the cup-stacked type CNTs, their combination are noncovalent combinations, which are relatively weak, so the nutrient contents can be fed to cells.

After the cell culture, the cup-stacked type CNTs may be heated at high temperature so as to reuse them.

Successively, another embodiment of the cell culture carrier of the present invention will be explained.

The cell culture carrier of the present embodiment is made of a carbon composite body including cup-stacked type CNTs, the carbon composite body is made by baking a mixed material including the cup-stacked type CNTs and resin so as to carbonize, and a surface of the carbon composite body is treated so that parts of the cup-stacked type CNTs are exposed in the surface.

The suitable resin is phenol resin, but it is not limited thereto. For example, the mixed material including the cup-stacked type CNTs and the resin is formed into a plate shape, then baked. The baking process is performed in an inert gas atmosphere, e.g., argon gas atmosphere, so as to carbonize the resin.

By baking the mixed material including the cup-stacked type CNTs and the resin, a carbon composite body (carbon-carbon composite body) can be produced. Namely, the cup-stacked type CNTs are tied by the carbonized resin. Note that, in this state, surfaces of the cup-stacked type CNTs are covered with the carbonized resin.

Thus, the carbon composite body is heat-treated in the air (oxidizing atmosphere) at temperature of around 400-600° C. so as to burn (oxidize) the carbonized resin covering the surface of the carbon composite body, so that parts of the cup-stacked type CNTs can be exposed in the surface of the carbon composite body. In another case, parts of the cup-stacked type CNTs may be exposed by grinding the surface of the carbon composite body with a grind stone, sand paper, etc.

A content of the cup-stacked type CNTs should be increased, but a preferable content thereof is about 30-90 wt %. If it is greater than 90 wt %, strength of the baked carbon composite body (or tying strength of the cup-stacked type CNTs) is weakened and the shape of the carbon composite body cannot be maintained.

The cell culture carriers are fixed to parts of the incubator (a jig for cell culture), which contact the culture solution and the cells, so as to culture the cells.

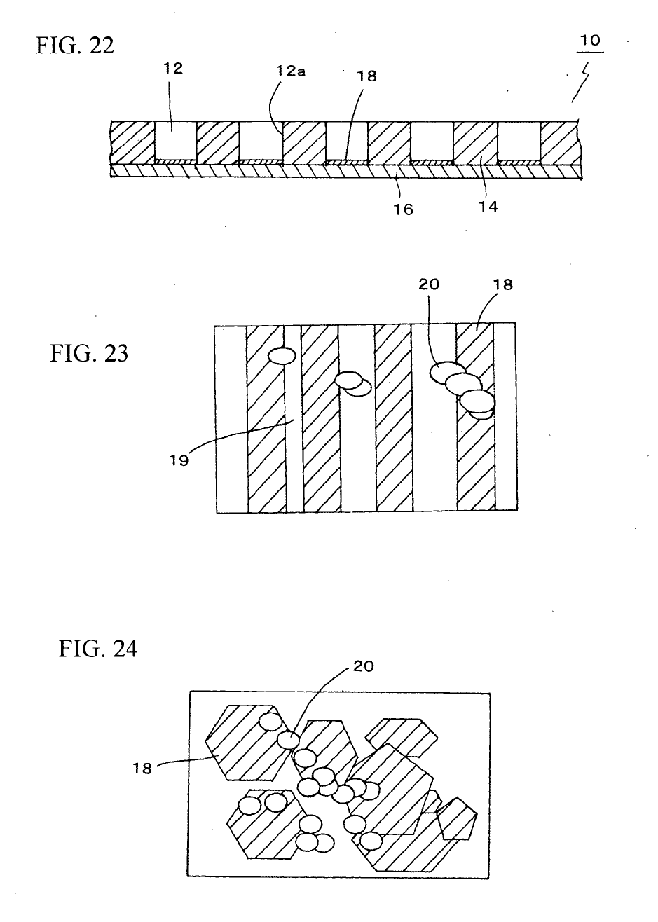

FIG. 22 shows an incubator (a jig) 110 having wells 112, which is an example of the jig.

The incubator 110 has plastic plates 114, whose thickness are about 1 cm and in which through-hole 112a for forming the wells 112 are formed, and a bottom plate 116 for closing bottoms of the through-hole 112a if the plastic plate 114.

The cell culture carriers 118 are respectively provided in the wells 112 and fixed therein by proper means.

If the cell culture carriers 118 are a flat plate, states of cultured cells cannot be inspected by light of microscope from a place under the bottom plate 116, so slits 119, whose widths are 0.05-0.5 mm, are formed in each of the cell culture carriers 118, as shown in FIG. 23, so as to gain visibility for the inspection by microscope, or the cell culture carriers 118 are formed like particles, as shown in FIG. 24, so as to inspect by microscope through gaps between the particle carriers 118. Note that, symbols 120 stand for cells.

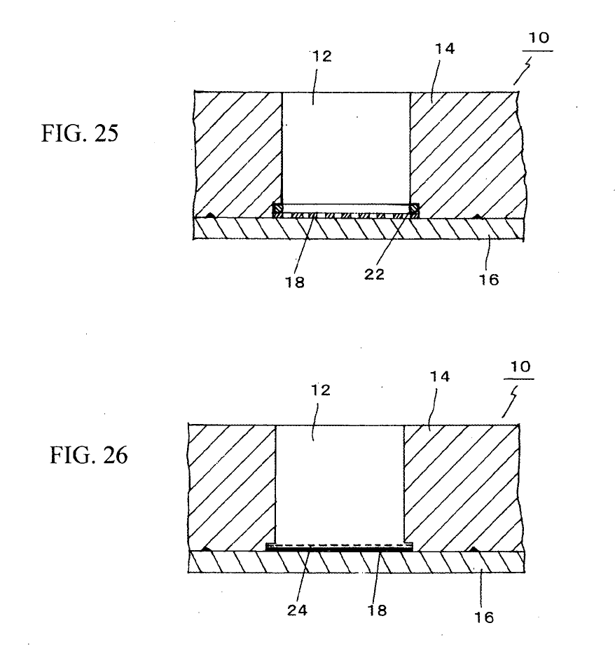

To fix the cell culture carriers 118 having the slits 119, which are shown in FIG. 23, in the wells 112, O-rings 122, for example, are respectively provided in the wells 112 so as to press edges of the carriers 118 and contact the plastic plates 114 and the bottom plate 116 as shown in FIG. 25, then the plastic plates 114 are fixed to the bottom plate 116 by welding or screws, so that the carriers 118 can be sealed and fixed.

To fix the particle-shaped carriers 118 shown in FIG. 24, in the wells 112, net-shaped pressing members 124, for example, are respectively provided in the wells 112, as shown in FIG. 26, so as to press the particle carriers 118, and an edge of each pressing member 124 is clamped between the plastic plates 114 and the bottom plate 116, and the plastic plates 114 are fixed to the bottom plate 116 by welding, so that the carriers 118 can be sealed and fixed.

In another case, the cell culture carriers 118 may be fixed to bottoms of the wells 112 by an adhesive.

By using the carriers 118 having the slits 119 or the particle-shaped carriers 118, the inspection by microscope can be performed, and area of each carrier 118 can be increased, so that area of a cell bonding part of each carrier 118 can be increased and cells can be effectively cultured.

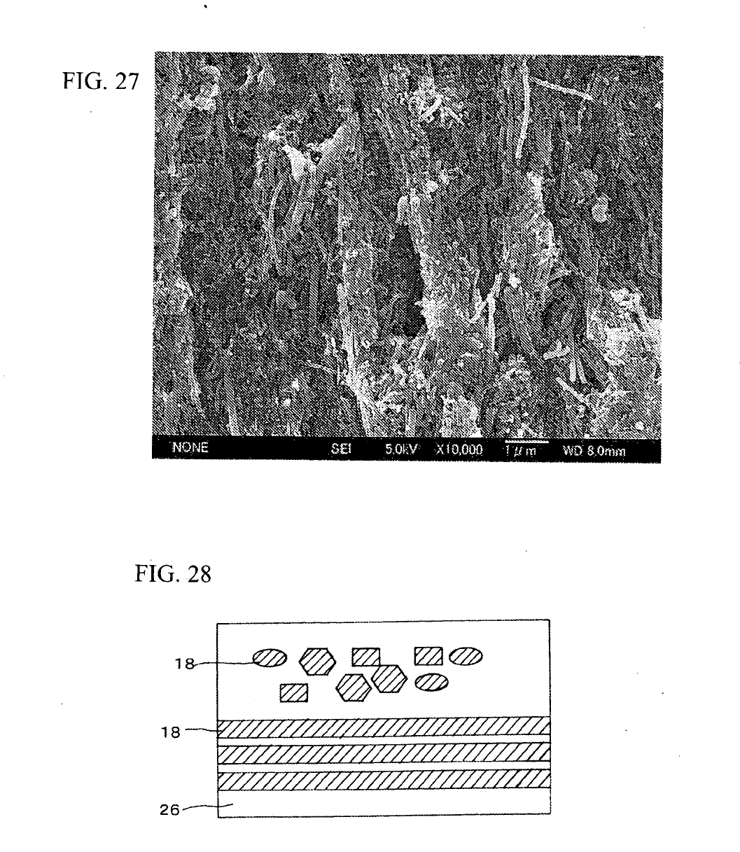

FIG. 27 is a scanning electron microscope of a surface of the cell culture carrier 118.

As described above, parts of the cup-stacked type CNTs can be exposed in the surface of the carbon composite body by heating and oxidizing the surface of the carbon composite body. The cup-stacked type CNTs are incorporated in the carbon composite body and headed at random. Therefore, some cup-stacked type CNTs expose their end hollow sections (shown by white outlines in FIG. 27) in the surface of the carbon composite body; if the cup-stacked type CNTs are obliquely incorporated, their surfaces are exposed so that edges of the carbon layers are exposed.

Very fine spaces (shown by black portions in FIG. 27) are formed between carbonized fine resin particles located between the cup-stacked type CNTs.

The cells 120 are cultured and grown on the cup-stacked type CNTs as scaffolds, which are exposed in the surface of the carriers 118.

In an ordinary cell culturing process, a culture solution is colored pink by a pigment, e.g., phenol red, and the color will changed to yellow with advancing the culture. The reason of changing the color to yellow is that metabolic wastes products (e.g., urea) are discharged from the cells to the culture solution; if the metabolic wastes products in the culture solution are increased, they make the cells difficult to incorporate nutrient contents so that the culture will be badly influenced.

In the case of using the carriers 118 of the present embodiment, the color of the culture solution was changed to colorless with advancing the culture.

The reason is that the metabolic wastes products derived from the cells were adsorbed and incorporated in the hollow sections of the cup-stacked type CNTs and the spaces between the carbonized fine resin particles, so that they were not mixed in the culture solution. Therefore, cells can be well cultured, proliferation rate can be increased, the cells can survive for a long time, and a period of using the cells can be made longer.

In comparison with the hollow sections of the cup-stacked type CNTs and the spaces between the carbonized fine resin particles, the cells are big so they are not incorporated therein. Further, the cup-stacked type CNTs are bonded and fixed by the carbonized resin particles, and they do not attach to the cells when the cells are separated, so that only the cells can be effectively used.

The separation can be easily performed by pipetting or an ordinary enzyme such as trypsin.

Note that, the incubator 110 can be reused. In the cell culture jig of the above described embodiment, the carriers 118, which have been baked and whose surfaces have been treated, are fixed in the wells; in other cases, the carriers 118 may be formed into rods or particles, they may be fixed on a plastic plate 126 or a film by thermocompression bonding, covering with a net or an adhesive, and the plastic plate 126 or the film may be fixed to a part of the jig, which contacts the culture solution and the cells. By fixing the carriers on the plastic plate 126 or the film, the carriers can be easily treated and attached without damaging the carriers.

In the above described embodiment, the well plate is used as the cell culture jig, further the carriers can be applied to other jigs for cell culture, e.g., flask, roller bottle.

In the above described embodiment, the cup-stacked type CNTs are used as the carriers, further fine powders of activated carbon may be used instead of the cup-stacked type CNTs.

Activated carbon has very fine holes. Size of an inlet of each very fine hole is relatively big, e.g., several μm (micron); each very fine hole is branched in an inner part, and size of the branched hole is much smaller, e.g., several nm. By finely crushing the activated carbon, size of the holes can be made several nm form the inlets.

A cell culture carrier can be produced by the steps of: mixing the fine powders of the activated carbon with resin; baking the mixed material until carbonizing the resin so as to form a carbon composite body; and treating a surface of the carbon composite body so as to expose parts of the activated carbon in the surface as well as the former embodiment.

Content of the activated carbon with respect to the mixed material is 30-90 wt % as well.

The surface of the carbon composite body may be heated and oxidized in an oxidizing atmosphere as well.

In the cell culture carrier made of the activated carbon, very fine holes of the activated carbon and fine spaces, which are formed between the carbonized resin particles, exist in the surface, so that metabolic wastes products derived from cells can be adsorbed and cannot be mixed in the culture solution, therefore the cells can be effectively cultured.

(EXPERIMENT 1)

The cell culture carrier shown in FIG. 12, in which the cup-stacked type CNTs are three-dimensionally entangled, was accommodated in an cell culture plate (not shown), and a culture solution was supplied into the cell culture plate until the carrier is soaked therein. The culture solution was an ordinary solution, which included 90 vol % of Williams medium E including PSN antibacterial agent and 10 vol % of FBS (fetus blood serum of bovine).

Liver parenchymal cells of a matured mouse were disseminated on the carrier, the carrier was heated in the incubator, in which temperature was about 37° C. and concentration of a carbon dioxide gas was about 5%, for 24 hours so as to culture and grow the liver cells, so that the liver cells three-dimensionally grew, as multiple layers, in spaces between the cup-stacked type CNTs, which acted as scaffolds of the culture.

(EXPERIMENT 2)

Cup-stacked type CNTs (sample names: 24-OX), which were heat-treated in the air for one hour at temperature of 520-530° C. so as to expose edges of carbon layers, and cup-stacked type CNTs (sample names: 24-OXSL), which were further ground for one hour by ball-milling, were used. A known culture solution RPM1164, to which 5 wt % of FBS was added, was used.

Samples were put in conical flask and hot-air-sterilized for four hours at temperature of 200° C. Then, the culture solution was added until concentration of the samples reached 10 g/1000 ml, and the samples were stored in a refrigerator. These basic solutions were diluted to use.

Molt-4 (cancerated lymphoblast) cells, which were very sensitive and induced apoptosis, were used as cells to be cultured.

24 well microplates were used as incubators.

The Molt-4 cells were inoculated and cultured in four solutions: (1) no cup-stacked type CNTs were included; (2) concentration of the cup-stacked type CNTs was 0.1 g/1000 ml; (3) concentration of the cup-stacked type CNTs was 1 g/1000 ml; and 0 concentration of the cup-stacked type CNTs was 10 g/1000 ml. Number of inoculating the cells was 50×10⁴ cell/well, and the cells were cultured under humidity supersaturation conditions, in which temperature were 37° C. and Co₂ concentrations were 5%.

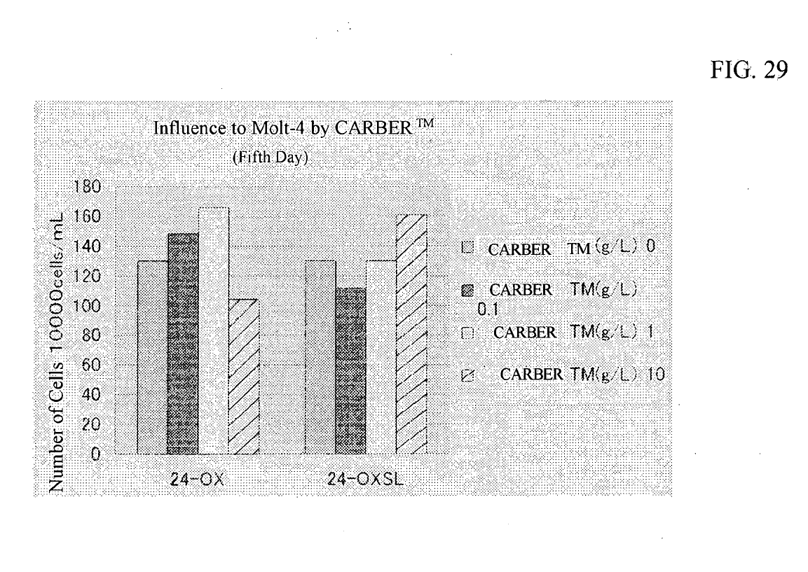

FIG. 29 is a graph of number of the cells after a lapse of five days. The cells were increased in all of the solutions; as to the samples 24-OX, the cells cultured in the solutions including the cup-stacked type CNTs, except the solution whose concentration of the cup-stacked type CNTs was 10 g/1000 ml, were increased much more than the cells cultured in the solutions including no cup-stacked type CNTs. In the drawing, the word “CARBERE” is a registered trademark.

On the other hand, as to the samples 24-OXSL, the cells cultured in the solution, whose concentration of the cup-stacked type CNTs was 10 g/1000 ml, were increased much more than the cells cultured in the solutions including no cup-stacked type CNTs.

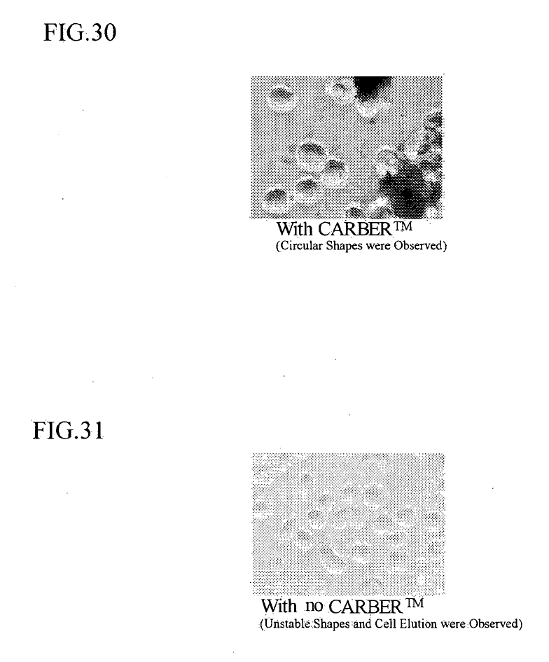

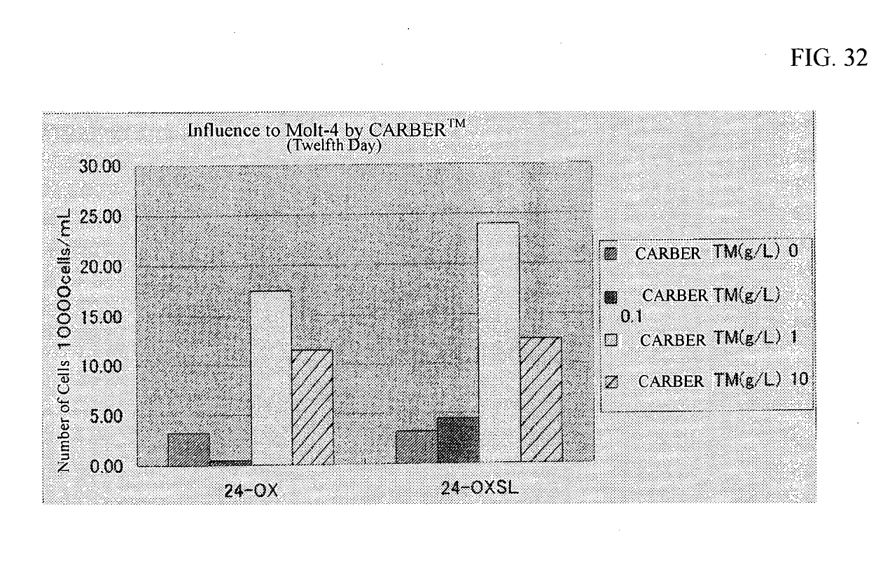

FIG. 30 is a micrograph of cells, which were the sample 24-OXSL, after a lapse of five days (black portions are the cup-stacked type CNTs); and FIG. 31 is a micrograph of cells, which were cultured in the culture solution including no cup-stacked type CNTs, after a lapse of five days. By adding the cup-stacked type CNTs, the cells stably maintained spherical shapes, and colors of cellular cytoplasm were stable. On the other hand, in the solutions including no cup-stacked type CNTs, some cells were deformed, cellular cytoplasm came out from the cells, and cell activity of the cultured cells were low. FIG. 32 is a graph of number of the cultured cells after a lapse of 12 days. Number of the cells cultured in the solution, whose concentration of the cup-stacked type CNTs was 0.1 g/1000 ml, was nearly equal to those of the cells cultured in the solutions including no cup-stacked type CNTs; in the solutions whose concentration of the cup-stacked type CNTs were 1 g/1000 ml and 10 g/1000 ml, reducing cells was stopped, and numbers of the living cells were five to ten times greater. Especially, as to the samples 24-OXSL, the living cells were remarkably observed in the solution whose concentration of the cup-stacked type CNTs was 1 g/1000 ml. The cells easily bonded on short cup-stacked type CNTs, which were shortened by, for example, grinding, as the scaffolds. Harmful metabolic wastes products discharged from the cells were adsorbed by the cup-stacked type CNTs, on which the cells bonded, and they are removed from the cells so that the cells can easily survive.

(EXPERIMENT 3)

Molt-4 (cancerated lymphoblast) cells, which were very sensitive and induced apoptosis, were used as cells to be cultured.

The carriers were made of mixed materials including resin, which respectively included 40 wt % and 80 wt % of the cup-stacked type CNTs.

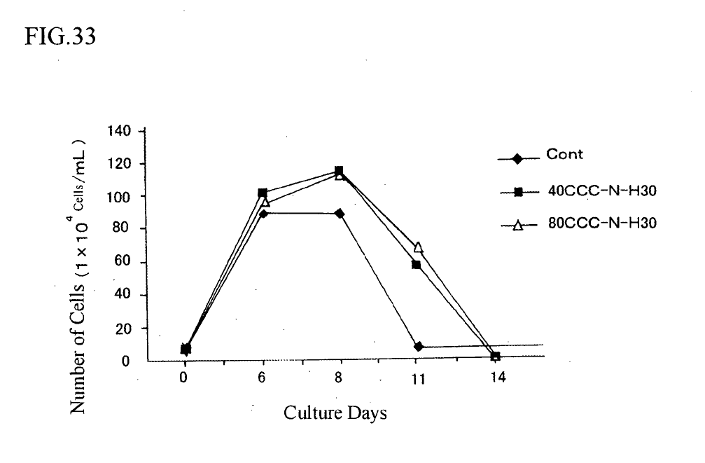

Culture solutions were serumless culture solutions KBM450 (produced by Kohjin-Bio Co. Ltd.) or RPMI solutions, to which 10 wt % of FBS was added; 24-wellplate incubators were used; number of the cells was 10×10⁴ cell/well; the cells of 2 ml/well were accommodated in each well; and the cells were cultured at temperature of 37° C. and Co₂ concentration of 5%. The cells were stained to trypan blue, and their number and survival rate were measured, by a blood cell counter, on the sixth, eighth, eleventh and fourteenth days. The results are shown in FIG. 33.

In FIG. 33, “Cont” indicates a case of culturing with conventional carrier without using the carrier 118; “40CCC-N-H30” and “80CCC-N-H30” indicate cases of culturing with the cup-stacked type CNTs whose contents were 40 wt % and 80 wt %, wherein the cells were cultured on the carriers oxidized at temperature of 500° C. without grinding treatment.

According to FIG. 33, numbers of the cells cultured with the carriers 118 were 20% greater than that of the cells cultured with the conventional carrier. Further, the cells cultured with the cup-stacked type CNTs survived two to three days longer than the cells cultured with the conventional carrier.

Note that, the cells were suitably separated from the carriers.

### EFFECTS OF THE INVENTON

The present invention is capable of growing multi-layered cells similar to cells of living organisms.

Further, the present invention is capable of growing cells, with a high density, for a long time.

In the present invention, the cup-stacked type CNT or the activated carbon is fixed to the cell culture carrier, the cup-stacked type CNT or the activated carbon has the spaces or the fine holes, and the fine holes are formed between the carbonized resin particles, which bond the cup-stacked type CNT or the activated carbon, so that metabolic wastes products discharged from the cells can be adsorbed in the spaces, etc., the cells can be suitably cultured without incorporating the metabolic wastes products therein, further the cells can survive for a long time and can be used for a long period.

Since the cup-stacked type CNT or the activated carbon is fixed in the carbonized resin, the cultured cells can be suitably separated.

What is claimed is:

1. A cell culture carrier comprising cup-stacked type carbon nano tubes produced by a catalytic chemical vapor deposition method, wherein a plurality of bottomless cup-shaped carbon layers (graphene sheet) are stacked in each of said cup-stacked type carbon nano tubes, and edges of the stacked carbon layers are exposed.

2. The cell culture carrier according to claim 1, wherein said cup-stacked type carbon nano tubes are entangled in a three dimensional space.

3. The cell culture carrier according to claim 1, wherein each of said cup-stacked type carbon nano tubes has a hollow shape having no bridge.

4. The cell culture carrier according to claim 3, wherein the edges of the carbon layers, which constitute an inner face and an outer face of the hollow shape of said cup-stacked type carbon nano tube, are exposed.

5. The cell culture carrier according to claim 1, wherein area of exposing the edges of the carbon layers is 2% or more of area of an outer face of said cup-stacked type carbon nano tube.

6. The cell culture carrier according to claim 1, wherein the exposed edges of the carbon layers are irregularly formed, and a part of a surface of said cup-stacked type carbon nano tube, in which the edges of the carbon layers are exposed, includes asperities at the atomic level.

7. The cell culture carrier according to claim 1, wherein said up-stacked type carbon nano tubes are not graphitized even if they are heat-treated at temperature of 2,500° C. or more.

8. The cell culture carrier according to claim 1, wherein a D-peak (1360 cm⁻¹) of a raman spectrum does not disappear even if said up-stacked type carbon nano tubes are heat-treated at temperature of 2,500° C. or more.

9. A cell culture carrier being made of a carbon composite body including cup-stacked type carbon nano tubes, wherein said cup-stacked type carbon nano tubes are produced by a catalytic chemical vapor deposition method, said carbon composite body is made by baking a mixed material including said cup-stacked type carbon nano tubes, in each of which a plurality of bottomless cup-shaped carbon layers are stacked and edges of the stacked carbon layers are exposed in a surface, and resin so as to carbonize, and a surface of the carbon composite body is treated so that parts of said cup-stacked type carbon nano tubes are exposed in the surface.

10. The cell culture carrier according to claim 9, wherein the surface of said carbon composite body is heated and oxidized in an oxidizing atmosphere so as to expose said cup-stacked type carbon nano tubes in the surface.

11. The cell culture carrier according to claim 9, wherein content of said cup-stacked type carbon nano tubes with respect to said mixed material is 30-90 wt %.

12. The cell culture carrier according to claim 11, wherein the surface of said carbon composite body is heated and oxidized in an oxidizing atmosphere so as to expose said cup-stacked type carbon nano tubes in the surface.

13. A cell culture carrier being made of a carbon composite body, wherein said carbon composite body is made by baking a mixed material including fine activated carbon powders and resin so as to carbonize the resin, and a surface of said carbon composite body is treated so that a part of the activated carbon is exposed in a surface.

14. The cell culture carrier according to claim 13, wherein the surface of said carbon composite body is heated and oxidized in an oxidizing atmosphere so as to expose the activated carbon in the surface.

15. The cell culture carrier according to claim 13, wherein content of the activated carbon with respect to said mixed material is 30-90 wt %.

16. The cell culture carrier according to claim 15, wherein the surface of said carbon composite body is heated and oxidized in an oxidizing atmosphere so as to expose the activated carbon in the surface.

17. A jig for cell culture comprising a cell culture carrier, wherein said cell culture carrier is made of a carbon composite body including cup-stacked type carbon nano tubes, said cup-stacked type carbon nano tubes are produced by a catalytic chemical vapor deposition method, said carbon composite body is made by baking a mixed material including said cup-stacked type carbon nano tubes, in each of which a plurality of bottomless cup-shaped carbon layers are stacked and edges of the stacked carbon layers are exposed in a surface, and resin so as to carbonize, a surface of the carbon composite body is treated so that parts of said cup-stacked type carbon nano tubes are exposed in the surface, and said cell culture carrier is fixed to a part, which contacts a culture solution and cells.

18. A jig for cell culture comprising a cell culture carrier, wherein said cell culture carrier is made of a carbon composite body, wherein said carbon composite body is made by baking a mixed material including fine activated carbon powders and resin so as to carbonize the resin, a surface of said carbon composite body is treated so that a part of the activated carbon is exposed in a surface, and said cell culture carrier is fixed to a part, which contacts a culture solution and cells.
